(19) United States
(12) Patent Application Publication
(10) Pub. No.: US 2024/0023854 A1
(43) Pub. Date: Jan. 25, 2024

(54) Laminated Blood Test Strip

(71) Applicant: Claire Kraft-Marsh, Clayton, NC (US)

(72) Inventor: Claire Kraft-Marsh, Clayton, NC (US)

(21) Appl. No.: 17/872,509

(22) Filed: Jul. 25, 2022

(51) Int. Cl. A61B 5/15; A61B 5/157

(52) U.S. Cl. CPC A61B 5/150358; A61B 5/157; A61M 2205/6072

(57) ABSTRACT

PKU pro is the only product of its kind that adds increased accuracy to assist with proper functionality for PKU cards, by not allowing the blood to go outside the designated circle area which is achieved through the use of a laminated area outside of each perimeter. This unprecedented products is uniquely designed with medically safe lamination film, durable materials to ensure long-term sustainability and, ultimately, serves to ensure a time efficient and safe means of conducting PKU tests for kids.

### BACKGROUND

While completing a phenylketonuria (PKU) test, health care professionals have to fill each relatively large circle on a test card, in succession perfectly, with blood taken through a prick in a baby's heel, without letting the blood bleed through the boundary of the circle. The process can be very error prone, particularly if the baby is, understandable, trying to move and/or is crying hysterically during the recording which to any event the consequence of the blood going beyond the intended area is that nurses or healthcare professionals are required to restart the entire process which can be incredibly time-consuming and challenging. There have been no products available as original equipment or as an aftermarket to address this problem.

An apparatus to simplify the process of completing a phenylketonuria (PKU) test for an infant is not being met by any known device or system at present. There have been no products available as original equipment or as an aftermarket to address this problem either.

### SUMMARY OF THE INVENTION

The main purpose of the laminated blood test strip is to provide users with updated laminated PKU test cards that prevent any excess blood from going beyond the intended control area thereby ruining the sample.

A disclosed test card includes a blood test strip laminated on a bottom portion of the test card and configured to define a plurality of blood circles free of the lamination on a top portion of the test card configured to receive a blood sample therein. The disclosure also includes an identifying indicia configured with information linking the blood sample with a donor of the blood sample. The disclosure further includes a chemical treatment of the test card proximal the plurality of blood circles.

### BRIEF DESCRIPTION OF THE DRAWINGS

FIG. 1 is a front elevational view of the laminated blood test strip in accordance with an embodiment of the present disclosure.

FIG. 2 are top views of a test strip seal and sealing patches in accordance with an embodiment of the present disclosure.

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

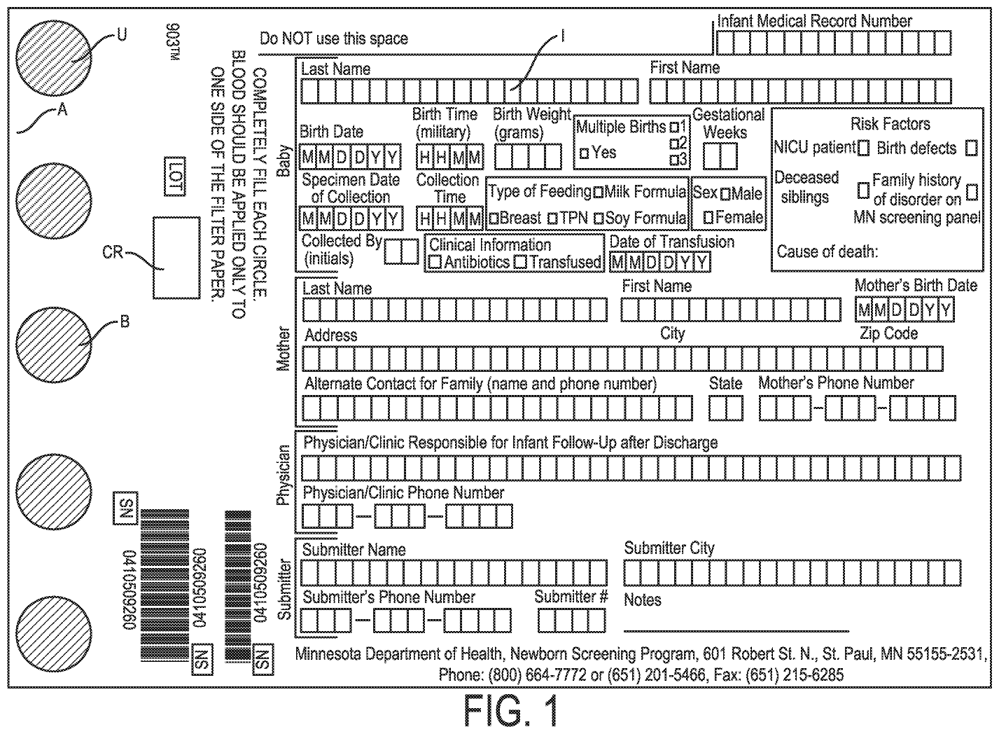

FIG. 1 is a front elevational view of the laminated blood test strip showing: clear or tinted laminated PKU test card referenced as A, and un-laminated circles for blood samples (ink free) referenced as B, in accordance with an embodiment of the present disclosure. The portions cross hatched bottom to top indicate an area or portion of the test card treated with a chemical as define herein for treating the blood sample. The indicia I includes personal information, a bar code and a CR (quick response QR) code thereon. The indicia I and/or the bar code and QR code link the blood samples in the blood circles with the blood donor patient including a baby, adult, child and minor. The blood circles B therefore are different diameters for varying amounts of blood deposited. Graduated circular areas are therefore included in embodiments.

The present disclosed laminated blood test strip, also known as the “PKU Pro” offers a modern accessory that improves the existing design of typical PKU cards while providing increased accuracy to assist with proper administration by not allowing the blood to go outside the designated circle area. PKU pro introduces a novel PKU test card where the primary blood holding circles/indicator spots on the card possess lamination around them. This is designed the reduce the repetitive nature of the existing test if the blood is not exactly inside the circle area as the control factor minimizes the likelihood of error normally associated with PKU testing conducted by medical staff. To use PKU pro, medical personnel would simply prick the baby's foot and allow releasing blood to drip into an unlaminated circle area which guarantees precision every time as the outer circumference is securely laminated preventing the fluid from moving beyond its housing area on the card. This innovative, top-quality product enhances the functionality of average PKU test cards and serves to allow for an improved testing experience and testing process, at all times.

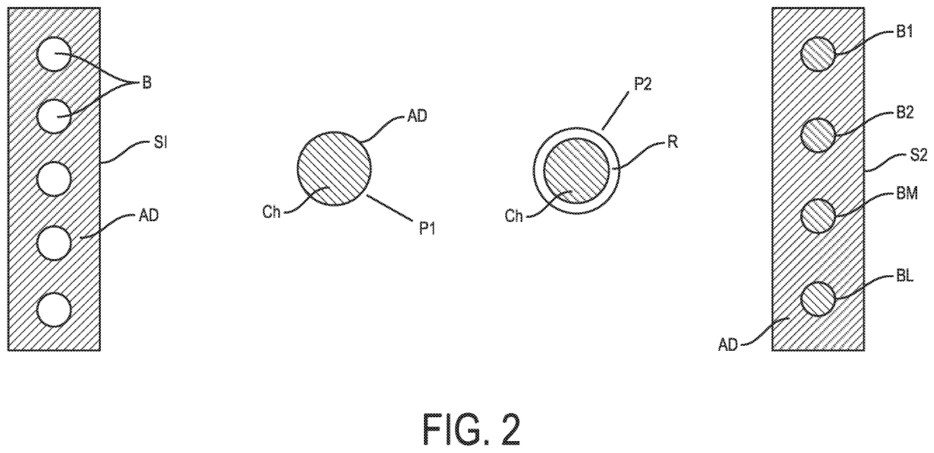

FIG. 2 are top views of a test strip seal and sealing patches in accordance with an embodiment of the present disclosure. The sealing strip S includes an adhesive AD on an underside thereof but not including areas matching with the blood circles B. The first patch P1 is circular or non-circular but large enough to cover a blood circle and include a chemical a for treating the blood sample on an underside AD (cross hatched from top to bottom) thereof. The second patch P2 is similar to the first patch P1 but includes a raised perimeter R for allowing the blood sample to pool there beneath. Strip S2 defines blood circles of graduated diameters B1, B2, BM and BL for graduated blood samples volumes according to tests needed for infants, adults, children and minors respectively.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

What is claimed is:

1. A test card comprising:
a blood test strip laminated on a bottom portion of the test card and configured to define a plurality of blood circles free of the lamination on a top portion of the test card configured to receive a blood sample therein;
an identifying indicia configured with information linking the blood sample with a donor of the blood sample; and
a chemical treatment of the test card proximal the plurality of blood circles.

2. The test card of claim 1, further comprising an adhesive test strip configured to overlay the blood test strip and seal in the blood sample in the plurality of blood circles.

3. The test card of claim 1, further comprising an adhesive test strip configured to overlap the blood test strip and seal the respective blood sample therein but be free of adhesive over the plurality of blood circles.

4. The test card of claim 1, further comprising a plurality of patches configured to seal in the respective blood samples in the plurality of blood circles.

5. The test card of claim 1, further comprising a plurality of patches each having a raised perimeter configured to seal in a pool of the blood sample in the plurality of blood circles.

6. The test card of claim 1, wherein the chemical treatment of the test card proximal the plurality of blood circles varies from one blood circle to the next.

7. The test card of claim 1, wherein the chemical treatment of the test card proximal the plurality of blood circles comprises a blood stabilizer.

8. The test card of claim 1, wherein the chemical treatment of the test card proximal the plurality of blood circles comprises a blood preservative.

9. The test card of claim 1, wherein the chemical treatment of the test card proximal the plurality of blood circles comprises a blood grouping reagent including solutions that determine blood groups.

10. The test card of claim 1, wherein the chemical treatment of the test card proximal the plurality of blood circles comprises a PKU (pyruvate kinase) blood reagent.

11. The test card of claim 1, wherein the chemical treatment of the test card proximal the plurality of blood circles comprises a graduated PKU (phenylketonuria) blood reagent configured to determine PKU levels in the blood sample.

12. The test card of claim 1, wherein the lamination is impervious to blood cells and blood serum and blood platelets.

13. The test card of claim 1, wherein the identifying indicia includes a QR (quick response) code.

14. The test card of claim 1, wherein the identifying indicia includes a bar code.

15. The test card of claim 1, wherein the chemical treatment of the test card includes a blood proofing agent configured to prevent a leakage of the blood sample into the test card from the plurality of blood circles.
